(19) United States
(12) Patent Application Publication
(10) Pub. No.: US 2019/0328361 A1
(43) Pub. Date: Oct. 31, 2019

(54) ULTRASOUND IMAGING SYSTEM AND METHOD

(71) Applicant: General Electric Company, Schenectady, NY (US)

(72) Inventors: Menachem Halmann, Wauwatosa, WI (US); Cynthia Owen, Powhatan, AR (US); Peter Lysyansky, Haifa (IL); Mor Vardi, Haifa (IL); Carmit Shiran, Haifa (IL)

(21) Appl. No.: 15/965,121

(22) Filed: Apr. 27, 2018

(51) Int. Cl. A61B 8/00; G06T 3/40; A61B 8/08

(52) U.S. Cl. CPC A61B 8/469; G06T 3/4038; A61B 8/463; A61B 8/523; A61B 8/5284; G06T 7/0012; A61B 8/4245; A61B 8/4444; G06T 2207/10132; G06T 2207/30061; A61B 8/085

(57) ABSTRACT

An ultrasound imaging system and method acquires ultrasound image data from moving an ultrasound probe over a body of a person, automatically divides the ultrasound image data into segments of interest based on where the ultrasound image data was acquired, and displays a panoramic view of the ultrasound image data that includes two or more of the segments of interest with at least one of the segments of interest displayed as a video.

### FIELD

The subject matter disclosed herein relates generally to ultrasound imaging systems.

### BACKGROUND

Imaging systems generate image data representative of imaged bodies. Some imaging systems are not real-time diagnosis or examination modalities in that the image data from these types of systems is obtained, otherwise presented as images or videos at a later time (subsequent to acquisition of the image data), and then presented to an operator for examination.

Other imaging systems are real-time diagnosis or examination modalities in that the image data from these types of systems is obtained and presented for diagnosis or examination by the operator in real-time. For example, the image data of a body can be visually presented to the operator for diagnosis or other examination while the imaging system continues obtaining additional image data of the same body.

One issue with real-time imaging modalities is that operators may miss one or more items of interest in the image data during examination. An operator may manually control a component of the imaging system (e.g., an imaging probe) to acquire the image data while the same operator also is visually inspecting the image data to identify the items of interest, such as regions of the image data that may represent an infection or diseased portion of the imaged body. This can result in the operator missing one or more items of interest in the image data.

For example, imaging a relatively large organ, such as a lung, can be difficult in real-time imaging modalities such as ultrasound due to different parts of the organ being imaged and visible at different times. Additionally, the lung may be in near constant motion, with pathological items of interest (e.g., diseased, infected, or otherwise damaged areas) in different parts of the lung being visible at different times. As a result, the operator of the imaging system may not have the ability to see different moving parts of the lung at the same time, and can risk missing pathological items of interest.

### BRIEF DESCRIPTION

In one embodiment, a method includes acquiring ultrasound image data from moving an ultrasound probe over a body of a person, automatically dividing the ultrasound image data into segments of interest based on where the ultrasound image data was acquired, and displaying a panoramic view of the ultrasound image data that includes two or more of the segments of interest with at least one of the segments of interest displayed as a video.

In one embodiment, a system includes an ultrasound probe configured to acquire ultrasound image data while moving over a body of a person, and one or more processors configured to automatically divide the ultrasound image data into segments of interest based on where the ultrasound image data was acquired. The one or more processors also are configured to direct a display device to display a panoramic view of the ultrasound image data that includes two or more of the segments of interest with at least one of the segments of interest displayed as a video.

In one embodiment, a method includes acquiring ultrasound image data from longitudinally moving an ultrasound probe over a person, automatically dividing the ultrasound image data into segments based on where the ultrasound image data is acquired in the person, and displaying a panoramic view of the segments of the ultrasound image data. The panoramic view includes at least one of the segments of the ultrasound image data displayed as a video.

### BRIEF DESCRIPTION OF THE DRAWINGS

The inventive subject matter described herein will be better understood from reading the following description of non-limiting embodiments, with reference to the attached drawings, wherein below:

FIG. 1 is a schematic diagram of an ultrasound imaging system in accordance with one embodiment of the inventive subject matter described herein;

FIG. 2 illustrates a thoracic cavity of a person according to one example;

FIG. 3 illustrates one embodiment of an ultrasound probe of the ultrasound imaging system shown in FIG. 1;

FIG. 4 illustrates a flowchart of one embodiment of a method for obtaining and concurrently presenting both static and dynamic image data;

FIG. 5 illustrates one example of ultrasound image data of a lung and ribs of a person acquired with the ultrasound probe shown in FIG. 3 held in a sagittal orientation;

FIG. 6 illustrates one example of formation of a combined view of image data acquired of a lung and ribs of a person using the imaging system shown in FIG. 1 with the ultrasound probe shown in FIG. 3 held in a sagittal orientation;

FIG. 7 illustrates an additional portion of the combined view of image data acquired of a lung and ribs of a person using the imaging system shown in FIG. 1 with the ultrasound probe shown in FIG. 3 held in a sagittal orientation;

FIG. 8 illustrates an additional portion of the combined view of image data acquired of a lung and ribs of a person using the imaging system shown in FIG. 1 with the ultrasound probe shown in FIG. 3 held in a sagittal orientation;

FIG. 9 illustrates an additional portion of the combined view of image data acquired of a lung and ribs of a person using the imaging system shown in FIG. 1 with the ultrasound probe shown in FIG. 3 held in a sagittal orientation;

FIG. 10 illustrates an additional portion of the combined view of image data acquired of a lung and ribs of a person using the imaging system shown in FIG. 1 with the ultrasound probe shown in FIG. 3 held in a sagittal orientation;

FIG. 11 illustrates an additional portion of the combined view of image data acquired of a lung and ribs of a person using the imaging system shown in FIG. 1 with the ultrasound probe shown in FIG. 3 held in a sagittal orientation;

FIG. 12 illustrates another example of formation of a combined view of image data acquired of a lung and ribs of a person using the imaging system shown in FIG. 1 with the ultrasound probe shown in FIG. 3 held in a transverse orientation;

FIG. 13 illustrates an additional portion of the combined view of image data acquired of a lung and ribs of a person using the imaging system shown in FIG. 1 with the ultrasound probe shown in FIG. 3 held in a transverse orientation;

FIG. 14 illustrates an additional portion of the combined view of image data acquired of a lung and ribs of a person using the imaging system shown in FIG. 1 with the ultrasound probe shown in FIG. 3 held in transverse orientation;

FIG. 15 illustrates an additional portion of the combined view of image data acquired of a lung and ribs of a person using the imaging system shown in FIG. 1 with the ultrasound probe shown in FIG. 3 held in a transverse orientation; and

FIG. 16 illustrates one example of the combined view of image data shown in FIGS. 12 through 15 with graphical anatomical features overlaid or otherwise displayed with the image data.

### DETAILED DESCRIPTION

One or more embodiments of the inventive subject matter described herein provide imaging systems and methods that obtain real-time image data of a body and display a combined view of the image data representative of different portions of the body, with the combined view concurrently showing both dynamic and static image data. For example, the systems and methods can be used to image a body using ultrasound, and to present a panoramic view of the body with one or more portions of the body being shown with moving ultrasound image data (e.g., a video or cine) and one or more other portions of the same body being shown with static ultrasound image data (e.g., a still image). Alternatively, the combined view may show all dynamic image data. For example, the combined view may concurrently show dynamic image data of different intercostal areas of a person's lung. While the description herein focuses on the use of ultrasound image data and imaging lungs, not all embodiments are limited to ultrasound image data and/or imaging lungs. One or more embodiments may apply the same inventive techniques and technology to image data acquired using another imaging modality and/or to image data showing a body part or organ other than a lung.

At least one technical effect of the inventive subject matter described herein includes the improved presentation of real-time image data to an operator so that the operator can concurrently view different portions of an imaged body, with one or more portions of the body being shown with moving image data and other portions of the body optionally being shown with static image data. The concurrent display of different portions of the imaged body in this way can assist the operator in more accurately diagnosing one or more disease, infection, or damage states of the imaged body.

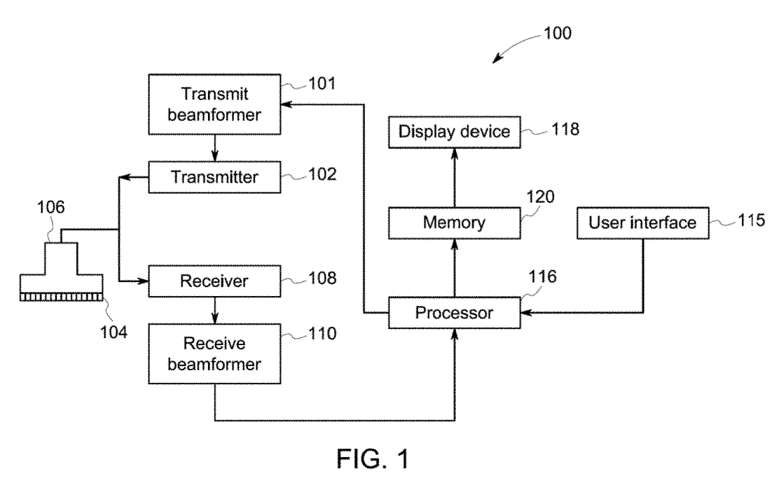

FIG. 1 is a schematic diagram of an ultrasound imaging system 100 in accordance with one embodiment of the inventive subject matter described herein. The ultrasound imaging system 100 includes a transmit beamformer 101 and a transmitter 102 that drive elements 104 within a probe 106 to emit pulsed ultrasonic signals into a body (not shown). According to an embodiment, the probe 106 may be a two-dimensional matrix array probe. Another type of probe capable of acquiring four-dimensional ultrasound data may be used according to one or more other embodiments. The four-dimensional ultrasound data can include ultrasound data such as multiple three-dimensional volumes acquired over a period of time. The four-dimensional ultrasound data can include information showing how a three-dimensional volume changes over time.

The pulsed ultrasonic signals are back-scattered from structures in the body, such as blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. The probe 106 may contain electronic circuitry to do all or part of the transmit and/or the receive beamforming. For example, all or part of the transmit beamformer 101, the transmitter 102, the receiver 108 and the receive beamformer 110 may be situated within the probe 106. Scanning may include acquiring data through the process of transmitting and receiving ultrasonic signals. Data generated by the probe 106 can include one or more datasets acquired with an ultrasound imaging system. A user interface 115 may be used to control operation of the ultrasound imaging system 100, including, to control the input of person data, to change a scanning or display parameter, and the like.

The ultrasound imaging system 100 also includes one or more processors 116 that control the transmit beamformer 101, the transmitter 102, the receiver 108 and the receive beamformer 110. The processors 116 are in electronic communication with the probe 106 via one or more wired and/or wireless connections. The processors 116 may control the probe 106 to acquire data. The processors 116 control which of the elements 104 are active and the shape of a beam emitted from the probe 106. The processors 116 also are in electronic communication with a display device 118, and the processors 116 may process the data into images for display on the display device 118. The processors 116 may include one or more central processors (CPU) according to an embodiment. According to other embodiments, the processors 116 may include one or more other electronic components capable of carrying out processing functions, such as one or more digital signal processors, field-programmable gate arrays (FPGA), graphic boards, and/or integrated circuits. According to other embodiments, the processors 116 may include multiple electronic components capable of carrying out processing functions. For example, the processors 116 may include two or more electronic components selected from a list of electronic components including: one or more central processors, one or more digital signal processors, one or more field-programmable gate arrays, and/or one or more graphic boards. According to another embodiment, the processors 116 may also include a complex demodulator (not shown) that demodulates the radio frequency data and generates raw data. In another embodiment, the demodulation can be carried out earlier in the processing chain.

The processors 116 are adapted to perform one or more processing operations according to a plurality of selectable ultrasound modalities on the data. The data may be processed in real-time during a scanning session as the echo signals are received, such as by processing the data without any intentional delay or processing the data while additional data is being acquired during the same imaging session of the same person. For example, an embodiment may acquire images at a real-time rate of seven to twenty volumes per second. The real-time volume-rate may be dependent on the length of time needed to acquire each volume of data for display, however. Accordingly, when acquiring a relatively large volume of data, the real-time volume-rate may be slower. Some embodiments may have real-time volume-rates that are considerably faster than twenty volumes per second while other embodiments may have real-time volume-rates slower than seven volumes per second.

The data may be stored temporarily in a buffer (not shown) during a scanning session and processed in less than real-time in a live or off-line operation. Some embodiments of the inventive subject matter may include multiple processors (not shown) to handle the processing tasks that are handled by the processors 116 according to the exemplary embodiment described hereinabove. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a volume-rate of, for example, ten to thirty hertz. Images generated from the data may be refreshed at a similar frame-rate. Other embodiments may acquire and display data at different rates. For example, some embodiments may acquire data at a volume-rate of less than ten hertz or greater than thirty hertz depending on the size of the volume and the intended application.

A memory 120 is included for storing processed volumes of acquired data. In one embodiment, the memory 120 is of sufficient capacity to store at least several seconds worth of volumes of ultrasound data. The volumes of data are stored in a manner to facilitate retrieval thereof according to its order or time of acquisition. The memory 120 may comprise any known data storage medium, such as one or more tangible and non-transitory computer-readable storage media (e.g., one or more computer hard drives, disk drives, universal serial bus drives, or the like).

Optionally, one or more embodiments of the inventive subject matter described herein may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters.

In various embodiments of the present invention, data may be processed by other or different mode-related modules by the processors 116 (e.g., B-mode, Color Doppler, M-mode, Color M-mode, spectral Doppler, Elastography, TVI, strain, strain rate, and the like) to form two- or three-dimensional image data. For example, one or more modules may generate B-mode, color Doppler, M-mode, color M-mode, spectral Doppler, Elastography, TVI, strain, strain rate and combinations thereof, and the like. The image beams and/or volumes are stored and timing information indicating a time at which the data was acquired in memory may be recorded. The modules may include, for example, a scan conversion module to perform scan conversion operations to convert the image volumes from beam space coordinates to display space coordinates. A video processor module may read the image volumes from a memory and displays an image in real time while a procedure is being carried out on a person. A video processor module may store the images in an image memory, from which the images are read and displayed.

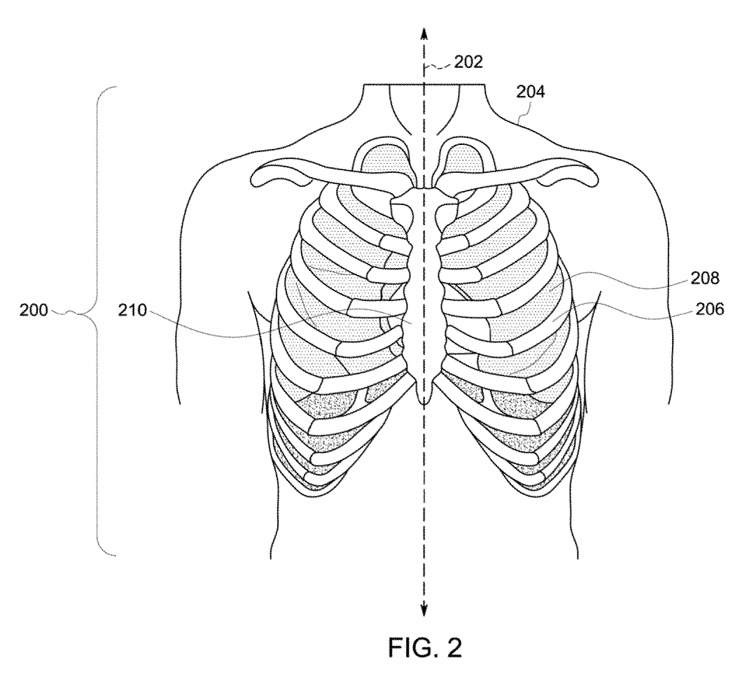

FIG. 2 illustrates a thoracic cavity 200 of a person 204 according to one example. The ultrasound image data that is obtained and used to train operators (as described herein) may represent portions of the thoracic cavity 200, including lungs 208, one or more ribs 206, and a sternum 210 of the person 204. In obtaining the ultrasound image data, the probe 106 shown in FIG. 1 may be held in contact with an exterior surface of the skin of the person 204 and moved longitudinally along the person 204 (e.g., in a direction that is closer to parallel to the length or height of the person 204 than one or more other directions). This movement also causes the probe 106 to transversely move relative to the ribs 206. For example, the probe 106 may be moved in a direction that is parallel or substantially parallel to the sagittal plane 202 of the person 204 (e.g., within ten degrees of parallel, within 15 degrees of parallel, etc.). As the probe 106 is moved in this direction during acquisition of ultrasound image data, the probe 106 moves transverse or substantially transverse to directions in which the various ribs 206 are elongated.

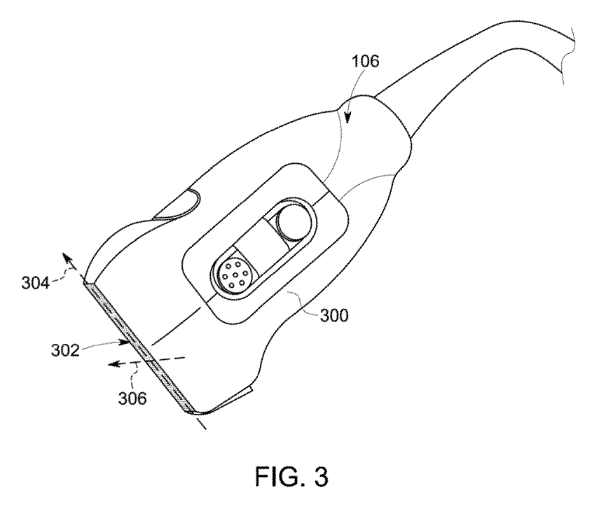

FIG. 3 illustrates one embodiment of the probe 106 of the ultrasound imaging system 100 shown in FIG. 1. The probe 106 can have a housing 300 that holds the drive elements 104 (not visible inside the housing 300 in FIG. 3). The housing 300 of the probe 106 interfaces (e.g., contacts) the person 204 along a face surface 302 of the housing 300. This face surface 302 is elongated along a first direction 304 relative to an orthogonal (e.g., perpendicular) direction 306.

The probe 106 can be moved along the outside of the person 204 along the thoracic cavity 200 to acquire ultrasound image data of the lungs 208 of the person 204. In one embodiment, the probe 106 is moved transversely to directions in which the ribs 206 are elongated. For example, the probe 106 can be moved along the exterior of the person 204 in directions that are more parallel to the sagittal plane 202 than perpendicular to the sagittal plane 202.

The probe 106 can be held in an orientation that has the elongated direction 304 of the housing 300 of the probe 106 oriented parallel to (or more parallel than perpendicular) the ribs 206 of the person 204 while the probe 106 is moved along the sagittal plane 202. This orientation of the probe 106 can be referred to as a sagittal position or orientation of the probe 106. Alternatively, the probe 106 can be held in an orientation that is perpendicular to the sagittal orientation. This orientation results in the probe 106 being oriented such that the elongated direction 304 of the housing 300 of the probe 106 is perpendicular to (or more perpendicular than parallel) the ribs 206 of the person 204 while the probe 106 is moved along the sagittal plane 202. This orientation of the probe 106 can be referred to as a transverse position or orientation of the probe 106.

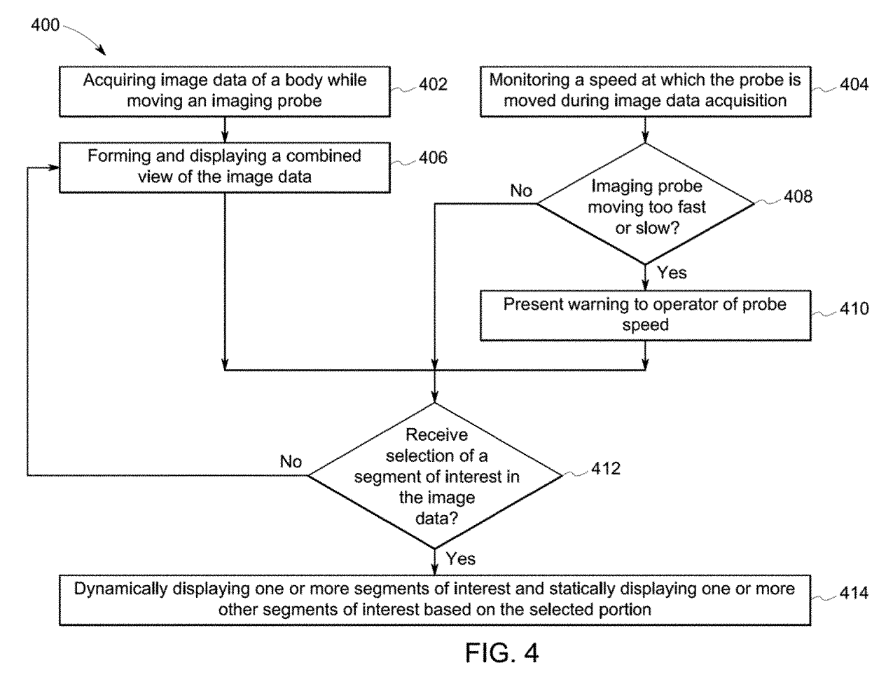

FIG. 4 illustrates a flowchart of one embodiment of a method 400 for obtaining and concurrently presenting dynamic image data and optionally static image data. The method 400 can represent operations performed by the ultrasound imaging system 100 to acquire ultrasound image data of a body (e.g., a lung or another body part) and to concurrently display dynamic (e.g., videos) portions of the image data of different areas of the imaged body, and optionally concurrently display static (e.g., still images) portions of the image data. This can assist the operator of the imaging system 100 to more easily see different parts of the imaged body at the same time using both still images and video. The imaging system 100 can change which portions of the image data are shown statically and/or dynamically. For example, the imaging system 100 can receive input from the operator and/or can automatically change which portions of an imaged lung are shown dynamically and, optionally, which other portions are shown statically.

Two or more operations and/or decisions of the method 400 can occur simultaneously (e.g., the operations and/or decisions begin and end at the same time) or concurrently (e.g., the operations and/or decisions begin and/or end at different times, but the time periods over which the operations and/or decisions are performed at least partially overlap each other). For example, the operations of 402 and 406 (acquiring image data, and forming and displaying a combined view of the image data, as described below) may be simultaneously and/or concurrently performed with one or more of the operations and/or decisions of 404, 408, and/or 410 (monitoring the speed of the probe 106 and potentially warning an operator that a probe is moving too fast or slow). Alternatively, these operations and/or decisions can be performed sequentially and not concurrently or simultaneously.

At 402, image data of a body is acquired while an imaging probe is moved. The image data can be ultrasound image data that is obtained by the probe 106 being moved along or over the body. In obtaining the ultrasound image data, the probe 106 may be held in contact with an exterior surface of the skin of the person 204 and moved transversely to the ribs 206. For example, the probe 106 may be moved in a direction that is parallel or substantially parallel to the sagittal plane 202 of the person 204 (e.g., within ten degrees of parallel, within 15 degrees of parallel, etc.). As the probe 106 is moved in this direction during acquisition of ultrasound image data, the probe 106 moves transverse or substantially transverse to directions in which the various ribs 206 are elongated. Alternatively, the probe 106 may be moved in directions that are parallel to the directions in which the ribs 206 are elongated.

In one embodiment, the ultrasound image data is acquired while the ultrasound probe 106 is held in the same orientation (e.g., only the sagittal orientation or only the transverse orientation) and moved in a single direction (e.g., only toward the head of the person 204 or only away from the head of the person 204). In another embodiment, the ultrasound image data is acquired while the ultrasound probe 106 is held in different orientations (e.g., part of the ultrasound image data is acquired while the probe 106 is held in the sagittal orientation and another part of the ultrasound image data is acquired while the probe 106 is held in the transverse orientation) and moved in a single direction. In another embodiment, the ultrasound image data is acquired while the ultrasound probe 106 is held in the same or different orientations and moved in two or more different directions (e.g., opposite directions, transverse directions, orthogonal directions, etc.).

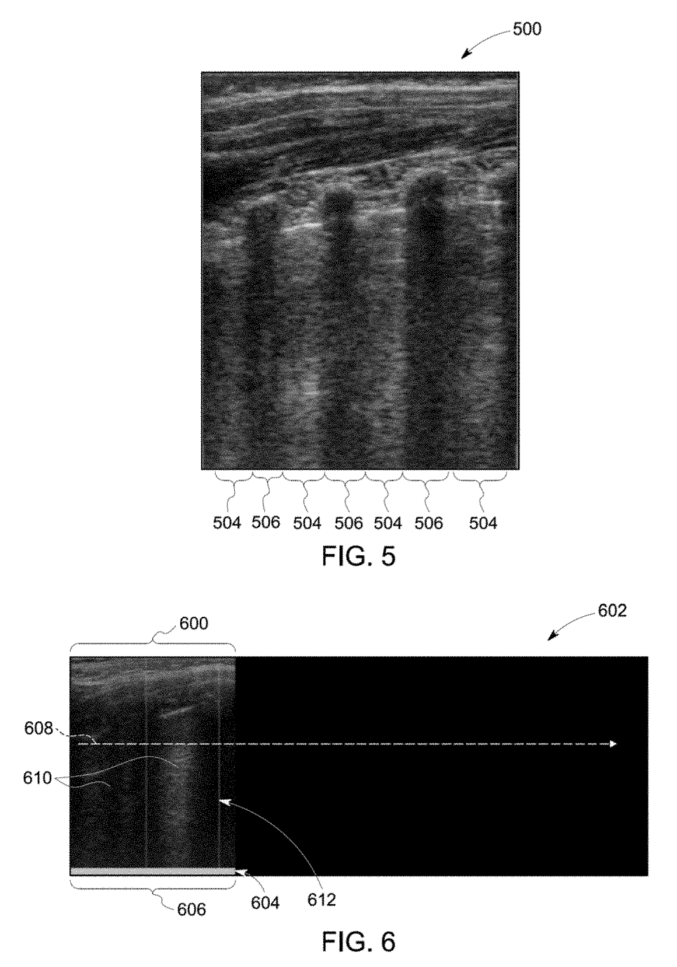

FIG. 5 illustrates one example of ultrasound image data 500 of the lung 208 and ribs 206 of the person 204 with the ultrasound probe 106 shown in FIG. 3 held in a sagittal orientation. This image data 500 can be acquired at 402 in the method 400. The ultrasound image data 500 shows a portion of an intercostal space 504 between ribs 206 of an unhealthy person. The image data 500 also shows parts of rib shadows 506 on either side of the intercostal space 504. These shadows 506 indicate where passage of the pulsed ultrasonic signals was blocked by the ribs 206.

Because the person 204 may be breathing as the image data 500 is acquired, the image data 500 may be a video or cine showing movement of one or more portions of the intercostal spaces 504 and/or rib shadows 506. This movement can result in one or more features of interest appearing at times and disappearing from the image data 500 at other times. For example, B-lines or other features in the image data 500 that indicate pneumonia infection, air bronchograms, or other damage may appear while the person 204 inhales, but may not be visible in the image data 500 while the person 204 exhales.

Returning to the description of the flowchart of the method 400 shown in FIG. 4, at 404, the speed at which the imaging probe is moved during image acquisition is monitored. As described above, this monitoring of the probe speed can occur at the same time that image data is acquired.

The processor 116 can examine the image data as the image data is acquired by the probe 106 to determine how quickly the probe 106 is moving relative to the body of the person 204. For example, as new or additional image data is acquired of new or different areas of the lung 208, ribs 206, or the like, the processor 116 can determine that the probe 106 is being moved. These new or different areas can include image data of additional intercostal spaces 504 and/or rib shadows 506. The processor 116 can determine that image data of additional intercostal spaces 504 and/or rib shadows 506 are being acquired based on changes in the characteristics of the image data, such as changes in brightness (e.g., increasing in brightness when an additional intercostal space 504 is being imaged or decreasing in brightness when an additional rib shadow 506 is being imaged), changes in color, etc.

The processor 116 can calculate a velocity at which the probe 106 is moved by dividing the estimated distance that the probe 106 is moved (e.g., based on how much image data of additional portions of the person 204 are acquired) by the time period over which the probe 106 is moved. Alternatively, the probe 106 can include one or more sensors, such as accelerometers, that can output data signals indicative of how rapidly the probe 106 is moving.

At 406, a combined view of the image data is formed and optionally displayed. The combined view of the image data can be a panoramic view of the image data. The combined view can be acquired by obtaining different portions of the image data as the probe 106 is moved over the person 204, and then stitching or otherwise combining these different image data portions together to form the combined view. The panoramic view can differ from other combined views of image data in that the image data acquired of different volumes or areas in the person 204 are shown alongside each other so as to not overlap each other. For example, the ultrasound image data acquired of first and second ribs 206 and the intercostal space between these first and second ribs 206 can be displayed in one part of the display device 118, the ultrasound image data acquired of second and third ribs 206 and the intercostal space between the second and third ribs 206 can be displayed in another part of the display device 118 (e.g., adjacent to or abutting the image data portion of the first and second ribs 206 and corresponding intercostal space), and so on.

The combined view of the image data can show or include more image data of the imaged body than the probe 106 can obtain. For example, the field of view or range of the imaged area by the probe 106 can be much smaller than the combined view. The processor 116 can obtain image data acquired while the probe 106 is at a first position or location relative to the person 204, additional image data acquired while the probe 106 is at a different, second position or location relative to the person 204, and so on. These different portions of the image data can then be combined to form the combined view of the image data.

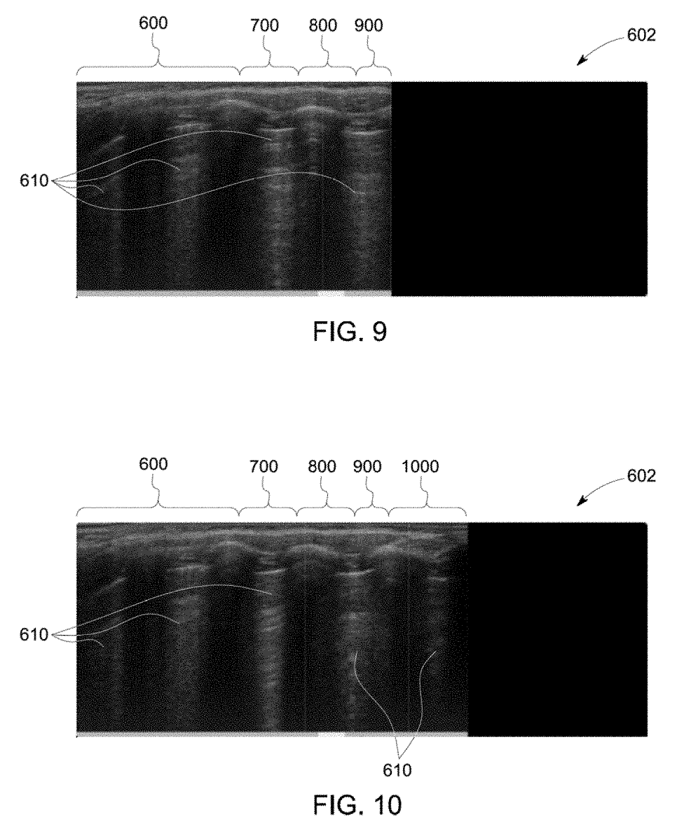

FIGS. 6 through 11 illustrate one example of formation of portions of a sagittal combined view 602 of image data acquired of a lung 208 and ribs 206 of the person 204 using the imaging system 100 with the ultrasound probe 106 shown in FIG. 3 held in a sagittal orientation. These Figures illustrate the sagittal combined view 602 as shown in a graphical user interface that can be presented to an operator of the imaging system 100 on the display device 118. The first portion 600 of the image data can be acquired (and optionally displayed on the display device 118) while the probe 106 is moved over the two lower or distal ribs 206 of the person 204. A second portion 700 of the sagittal combined view 602 of the image data can be acquired while the probe 106 moves over additional ribs 206 of the person 204 that are closer to the head of the person 204 than the ribs 206 shown in the first portion 600 of the sagittal combined view 602 of image data. A third portion 800 of the sagittal combined view 602 of the image data can be acquired while the probe 106 moves over additional ribs 206 of the person 204 that are closer to the head of the person 204 than the ribs 206 shown in the first and second portions 600, 700 of the sagittal combined view 602 of image data. A fourth portion 900 of the sagittal combined view 602 of the image data can be acquired while the probe 106 moves over additional ribs 206 of the person 204 that are closer to the head of the person 204 than the ribs 206 shown in the first, second, and third portions 600, 700, 800 of the sagittal combined view 602 of image data. A fifth portion 1000 of the sagittal combined view 602 of the image data can be acquired while the probe 106 moves over additional ribs 206 of the person 204 that are closer to the head of the person 204 than the ribs 206 shown in the first, second, third, and fourth portions 600, 700, 800, 900 of the sagittal combined view 602 of image data. A sixth portion 900 of the sagittal combined view 602 of the image data can be acquired while the probe 106 moves over additional ribs 206 of the person 204 that are closer to the head of the person 204 than the ribs 206 shown in the first, second, third, fourth, and fifth portions 600, 700, 800, 900, 1000 of the sagittal combined view 602 of image data.

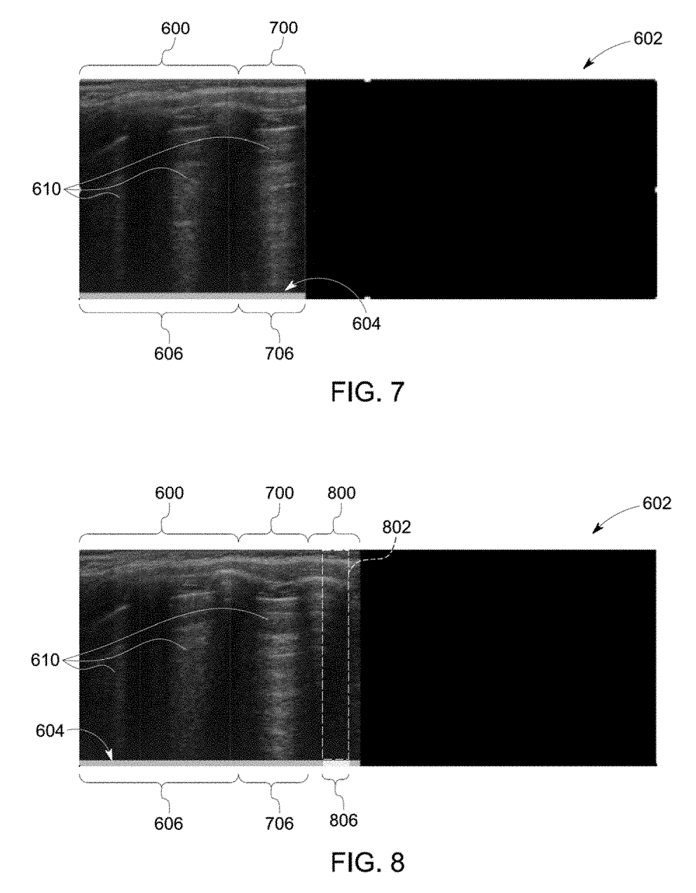

The different portions 600, 700, 800, 900, 1000, 1100 of the sagittal combined view 602 of the image data can be displayed on the display device 118 as the image data corresponding to the different portions 600, 700, 800, 900, 1000, 1100 are obtained. For example, once the image data representing the first portion 600 is obtained by the imaging probe 106, the first portion 600 can be displayed (as shown in FIG. 6). As the image data representing the second portion 700 is subsequently obtained by the imaging probe 106, the second portion 700 can be displayed alongside the first portion 600 (as shown in FIG. 7). As the image data representing the third portion 800 is subsequently obtained by the imaging probe 106, the third portion 800 can be displayed alongside the first and second portions 600, 700 (as shown in FIG. 8), and so on. In this way, the displayed image data can continue to increase in size (e.g., laterally to the right side in the view of FIGS. 6 through 11).

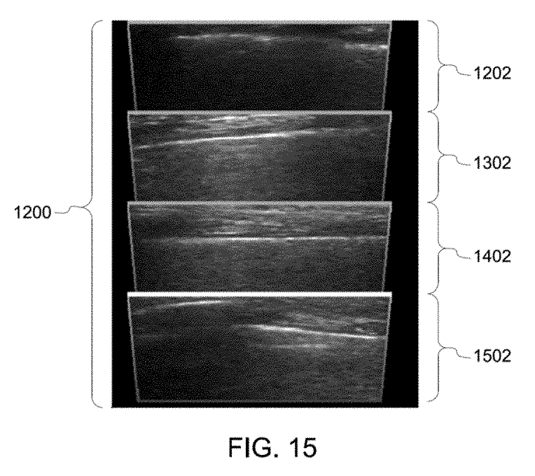

FIGS. 12 through 15 illustrate one example of formation of portions of a transverse combined view 1200 of image data acquired of a lung 208 and ribs 206 of the person 204 using the imaging system 100 with the ultrasound probe 106 shown in FIG. 3 held in a transverse orientation. These Figures illustrate the transverse combined view 1200 as shown in a graphical user interface that can be presented to an operator of the imaging system 100 on the display device 118. A first portion 1202 of the image data can be acquired (and optionally displayed on the display device 118) while the probe 106 is moved over the two lower or distal ribs 206 of the person 204. A second portion 1302 of the transverse combined view 1200 of the image data can be acquired while the probe 106 moves over additional ribs 206 of the person 204 that are closer to the head of the person 204 than the ribs 206 shown in the first portion 1202 of the combined view 1200 of image data. A third portion 1302 of the transverse combined view 1200 of the image data can be acquired while the probe 106 moves over additional ribs 206 of the person 204 that are closer to the head of the person 204 than the ribs 206 shown in the first and second portions 1202, 1302 of the transverse combined view 1200 of image data. A fourth portion 1402 of the transverse combined view 1200 of the image data can be acquired while the probe 106 moves over additional ribs 206 of the person 204 that are closer to the head of the person 204 than the ribs 206 shown in the first, second, and third portions 1202, 1302, 1402 of the transverse combined view 1200 of image data.

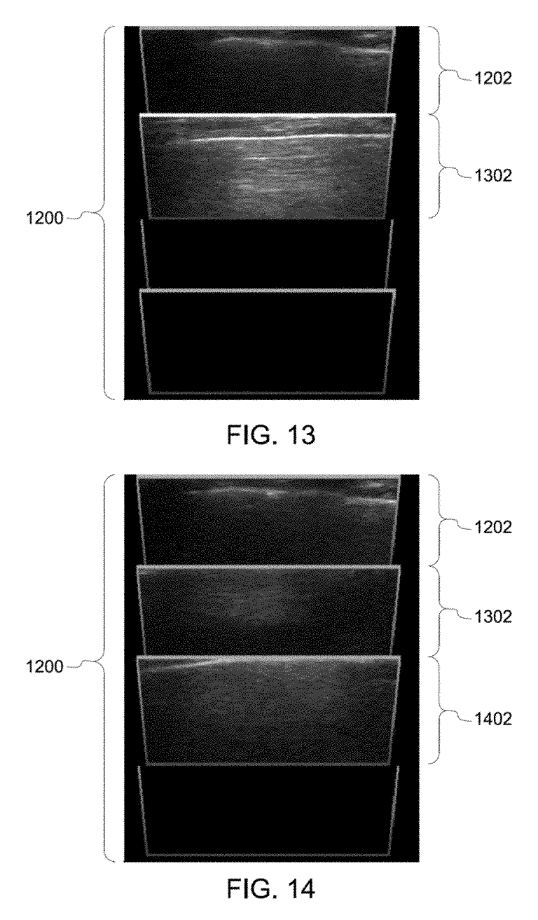

The different portions 1202, 1302, 1402, 1502 of the transverse combined view 1200 of the image data can be displayed on the display device 118 as the image data corresponding to the different portions 1202, 1302, 1402, 1502 are obtained. For example, once the image data representing the first portion 1202 is obtained by the imaging probe 106, the first portion 1202 can be displayed (as shown in FIG. 12). As the image data representing the second portion 1302 is subsequently obtained by the imaging probe 106, the second portion 1302 can be displayed alongside the first portion 1202 (as shown in FIG. 13). As the image data representing the third portion 1402 is subsequently obtained by the imaging probe 106, the third portion 1402 can be displayed alongside the first and second portions 1202, 1302 (as shown in FIG. 14), and so on.

Both the sagittal and transverse combined views 602, 1200 can be referred to as panoramic views of the ultrasound image data in that these views 602, 1200 combine image data acquired at different locations into a single static and/or moving image (or a combination thereof).

In one embodiment, forming the combined view of the image data can include automatically identifying segments of interest in the image data. A segment of interest can be a subset or portion of the combined image data that is selected based on characteristics of the image data. The processor 116 can examine characteristics of the pixels (or other subsets of the image data) to identify the segments of interest, such as the color, intensity, brightness, or the like, of the pixels in the image data.

As one example, the processor 116 can examine the pixels of the image data to automatically identify different intercostal spaces 504 as the different segments of interest. The processor 116 can monitor the brightness of pixels along one or more lines 608 (shown in FIG. 6) or other shapes in the image data as the image data is acquired. The line 608 can extend parallel to the direction in which the image data extends in the combined view as additional image data is obtained. The brightness of pixels along the line 608 will decrease in rib shadows 506 and increase in intercostal spaces 504. The processor 116 can use the changes in pixel intensity to identify different intercostal spaces 504, such as by determining when the pixel brightness along the line 608 decreases (indicating a rib shadow 506) and then increases (indicating an intercostal space). The processor 116 can use the identified intercostal spaces 504 to determine segments of interest 610 in the image data. As shown in FIGS. 6 through 11, the segments of interest 610 represent different intercostal spaces 504, and are separated from each other by boundaries 612 (shown in FIG. 6, but appearing in FIGS. 6 through 11), which may be visible on the display device 118 to aid the operator in viewing and/or selecting one or more segments of interest 610. The segments of interest 610 optionally can be referred to as inter-rib segments. The segments of interest 610 can be shown in the transverse combined view 1200 as separate portions 1202, 1302, 1402, 1502, as shown in FIGS. 12 through 15. For example, the portion 1202 can represent one segment of interest 610, the portion 1302 can represent another, different segment of interest 610, and so on.

The processor 116 optionally can synchronize the videos of image data in the combined view 602, 1200 for different segments of interest 610. The video image data of the different segments of interest 610 can be obtained at different times due to the movement of the probe 106 longitudinally along the person 204. As a result, the video image data corresponding to the different segments of interest 610 may show movement, but this movement may not be synchronized with each other due to the different segments of interest 610 being obtained at different times. For example, while one segment of interest 610 is showing movement of an intercostal space 504 during inhalation by the person 204, another segment of interest 610 may show movement of another intercostal space 504 during exhalation by the person 204. But, because the video for these different segments of interest 610 are displayed at the same time, one intercostal space 504 may be moving as though the person 204 is inhaling while another intercostal space 504 appears to be moving as though the person 204 is exhaling at the exact same time.

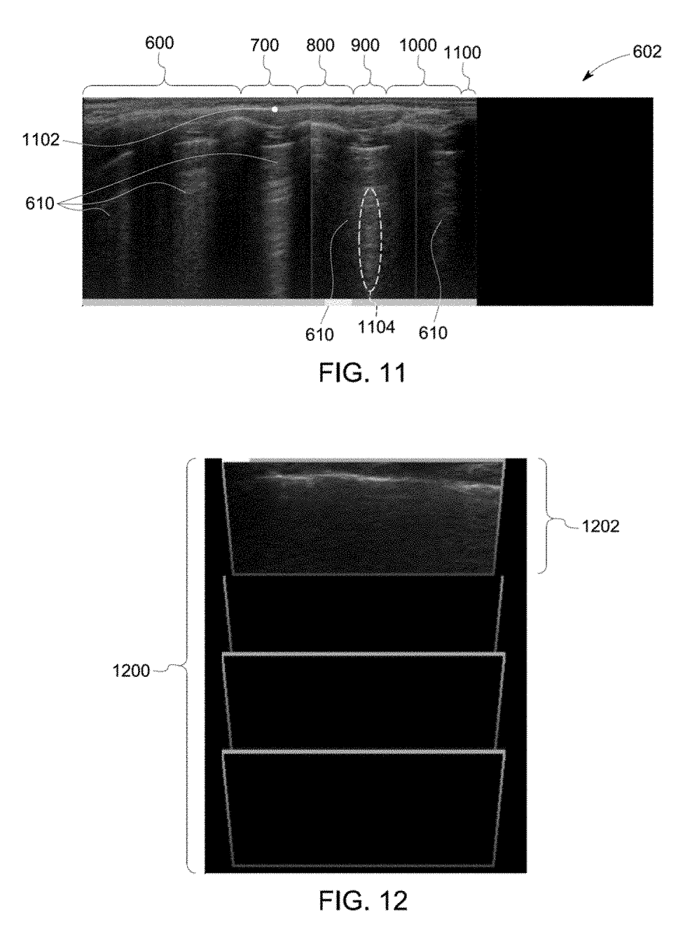

The processor 116 can synchronize the videos of the different segments of interest 610 based on respiratory cycle timing of the person 204 being imaged. The respiratory cycle can be measured or estimated by the processor 116 based on movement of one or more portions of the image data. For example, as shown in FIG. 11, a location 1102 in the sagittal combined view 602 of the image data may move (if included in a portion of the image data that is dynamically displayed, as described herein). This location 1102 can correspond to a pleura of the person 204 or another part of the person 204. This location 1102 may move laterally in the sagittal combined view 602 (e.g., left and right in the perspective of FIG. 11) and/or may move vertically in the sagittal combined view 602 (e.g., up and down in the perspective of FIG. 11). The speed and/or frequency at which the location 1102 moves back-and-forth in the sagittal combined view 602 can be measured by the processor 116 and used to estimate the respiratory rate of the person 204. For example, if the location 1102 moves back-and-forth at a frequency of twelve times per minute, then the processor 116 can determine that the respiratory cycle of the person 204 is twelve breaths per minute. Alternatively, a ventilator may be controlling the respiratory cycle of the person 204, and the processor 116 can receive a signal from the ventilator that reports the respiratory rate at which the person 204 is breathing.

The processor 116 can use the calculated, estimated, or reported respiratory rate or cycle to synchronize the video image data associated with the different segments of interest 610. For example, the processor 116 can direct the display device 118 to play the video image data associated with each segment of interest 610 in a repeating loop, with each repetition of the video loop starting at a common point in time in the respiratory cycle of the person 204. The processor 116 can direct the display device 118 to start the video of each segment of interest 610 at the beginning of each respiratory cycle of the person 204, at the beginning of each inhalation by the person 204, at the end of each exhalation by the person 204, or the like.

The processor 116 optionally can temporally scale the video image data for one or more of the segments of interest 610 based on a change in the respiratory cycle of the person 204. The person 204 may change how rapidly he or she breathes during acquisition of the image data in the sagittal combined view 602. For example, the image data of one segment of interest 610 may be obtained while the person 204 is breathing at a rate of twelve breaths per minute, while the image data of another segment of interest 610 may be obtained while the person if breathing at a faster or slower rate, such as twenty breaths per minute or six breaths per minute.

The processor 116 can monitor changes in the breathing rate of the person 204 and temporally extend or compact the video image data for one or more segments of interest 610 based on a change in the breathing rate. For example, the image data associated with a segment of interest 610 obtained while the person 204 was breathing at a slower rate may be temporally contracted or compacted by the processor 116 to extend over a shorter period of time. For example, the image data associated with another segment of interest 610 obtained while the person 204 was breathing at a faster rate may be temporally expanded or extended by the processor 116 to extend over a longer period of time. Contracting or expanding the image data can result in the video clips or portions of the image data for different segments of interest 610 to extend over the same period of time regardless of changes in the breathing rate of the person 204.

For example, the image data for a segment of interest 610 obtained while the person 204 was breathing at a rate of ten breaths per minute can be extended so that each breath of the person 204 (as represented by the video image data for that segment of interest 610) occurs over a repeating loop lasting eight seconds. The image data for another segment of interest 610 obtained while the person 204 was breathing at a rate of six breaths per minute can be contracted so that each breath of the person 204 (as represented by the video image data for that segment of interest 610) occurs over a repeating loop lasting the same eight seconds. This can allow for the video clips associated with each segment of interest 610 to begin and end at the same times.

In one embodiment, the processor 116 can re-arrange the layout of two or more of the segments of interest 610 in the displayed image data. The processor 116 can use a movement indication received from the operator of the imaging system as input to re-arrange which segments of interest 610 are adjacent to each other. For example, the operator can use a touchscreen of the display device 118 or another input device to select a first segment of interest 610 that is between second and third segments of interest 610. The input provided by the operator can then move the first segment of interest 610 to another location in the sagittal combined view 602, such as between second and third segments of interest 610 (or another location).

The processor 116 can automatically examine frames of the ultrasound image data for at least one of the segments of interest 610 to identify one or more regions of interest. The regions of interest can represent pathological structures or other items of interest in the image data. The pathological structures can represent infected, damaged, or diseased areas of a different body.

The processor 116 can examine characteristics of pixels in the sagittal combined view 602 of the image data to identify where the pathological structures are located without operator intervention. This can involve the processor 116 identifying a group of interconnected or neighboring pixels having an intensity, color, or other characteristic that is within a designated range of each other, and optionally where the average, median, or mode characteristic of the pixels in the group differs from pixels outside the group of pixels (e.g., by at least a threshold amount). For example, the processor 116 can identify boundaries between groups of pixels having different characteristics, with the group of pixels that is enclosed (e.g., by a closed perimeter of other group or groups of pixels) representing a pathological structure. In the example shown in FIG. 11, the processor 116 may identify B-lines in one of the intercostal spaces 504 as a region of interest 1104. This region of interest 1104 can indicate an infection cause by pneumonia or another disease state or damage.

The region of interests that are identified by the processor 116 may occur in frames at different times in the video image data associated with different segments of interest 610. For example, a first pathological structure may occur earlier in a video of a first segment of interest 610 than a second pathological structure in a video of a second segment of interest 610. The processor 116 can select the frame or frames in the image data for the segments of interest 610 that show the regions of interest, and display these frames as the segments of interest 610 in the sagittal combined view 602.

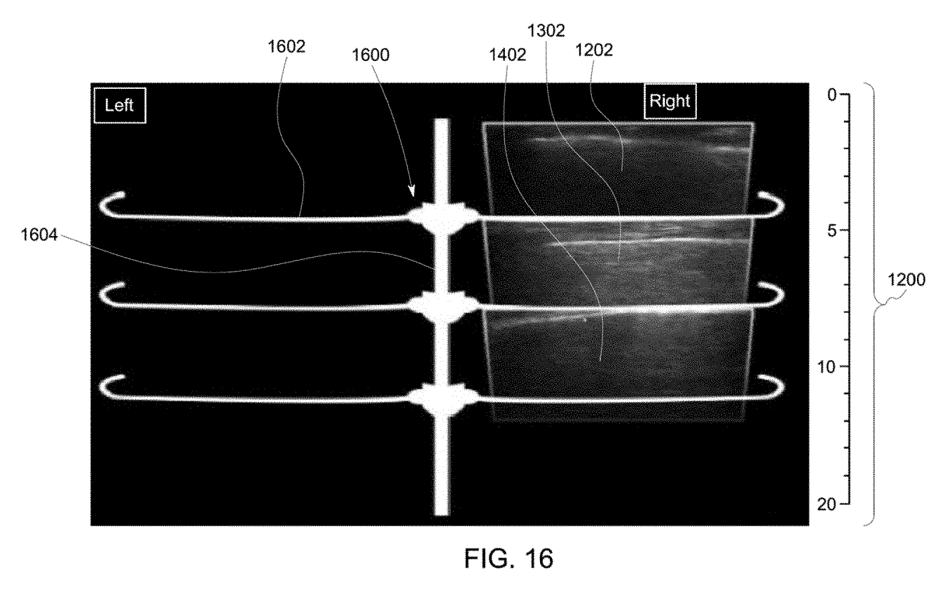

In one embodiment, the processor 116 can direct the display device 118 to display one or more graphical anatomical features onto or with the image data to assist the operator in visualizing where the different segments of interest 610 are located. FIG. 16 illustrates one example of the combined view 1200 of image data with graphical anatomical features 1600 overlaid or otherwise displayed with the image data. The graphical anatomical features 1600 can be a single icon or multiple icons, and can represent one or more anatomical bodies or features of the person 204. In the illustrated example, the graphical anatomical features 1600 represent the ribs 206 and the sternum 210 of the person 204. The graphical anatomical features 1600 include several rib lines 1602 that represent locations of the ribs 206 of the person 204 and a sternum line 1604 that represents the location of the sternum 210 of the person 204.

The processor 116 can direct the display device 118 to present the graphical anatomical features 1600 so that the rib lines 1602 are shown between the different segments of interest 610 in the image data (e.g., between the different portions 1202, 1302, 1402). Although not shown in FIG. 16, the processor 116 also can direct the display device 118 to present image data from both lungs of the person 204, with the combined image data obtained from the right lung 208 shown on the right (or left) side of the display device 118 and the combined image data obtained from the left lung 208 shown on the left (or right) side of the display device 118. The processor 116 can direct the display device 118 to show the sternum line 1604 between the combined image data of the right lung and the combined image data of the left lung. These graphical anatomical features 1600 can assist the operator in visualizing from where the different segments of interest 610 in the image data were or are acquired.

Returning to the description of the flowchart of the method 400 shown in FIG. 4, at 408, a determination is made as to whether the imaging probe is being moved too slow or too fast during acquisition of the image data. The speed at which the probe 106 is moved during acquisition of image data can be compared to one or more designated threshold speeds, such as a lower (e.g., slower) speed limit and an upper (e.g., faster) speed limit. If the processor 116 determines that the probe 106 is being moved faster than the upper limit or slower than the lower limit, then the processor 116 can decide to warn the operator to change the speed at which the probe 106 is being moved. Moving the probe 106 too quickly or too slowly can negatively impact the quality and/or quantity of the image data that is acquired in one or more locations of the person 204. If the probe 106 is moving too fast or too slow during image data acquisition, then flow of the method 400 can proceed toward 410. But, if the probe 106 is not moving too fast or too slow, then flow of the method 400 can proceed toward 412.

In one embodiment, the speed limits to which the probe speed is compared by the processor 116 can change based on one or more characteristics of the person 204. For example, different upper and/or lower speed limits can be used for persons 204 of different ages. Younger persons 204 may be imaged with a reduced upper speed limit (relative to older persons 204). As another example, the speed limits can change based on a disease state of the person 204. A person 204 having a disease or infection such as pneumonia, chronic obstructive pulmonary disease, etc., may have a slower upper speed limit (relative to persons 204 not having the same disease state).

As another example, the upper and/or lower speed limits can vary based on a respiratory cycle timing of the person 204 (e.g., the respiratory rate of the person 204). The upper and/or lower speed limits can be increased for persons 204 that breathe more rapidly, and can be reduced for slower breathing persons 204. The respiratory cycle can be measured or estimated by the processor 116 based on movement of one or more portions of the image data. For example, as shown in FIG. 11, a location 1102 in the sagittal combined view 602 of the image data may move (if included in a portion of the image data that is dynamically displayed, as described herein). This location 1102 may move laterally in the sagittal combined view 602 (e.g., left and right in the perspective of FIG. 11) and/or may move vertically in the sagittal combined view 602 (e.g., up and down in the perspective of FIG. 11). The speed and/or frequency at which the location 1102 moves back-and-forth in the sagittal combined view 602 can be measured by the processor 116 and used to estimate the respiratory rate of the person 204. For example, if the location 1102 moves back-and-forth at a frequency of twelve times per minute, then the processor 116 can determine that the respiratory cycle of the person 204 is twelve breaths per minute. Alternatively, a ventilator system may be controlling the respiratory cycle of the person 204, and the processor 116 can receive a signal from the ventilator system indicating the respiratory rate at which the person 204 is breathing.

If the probe 106 is being moved at a speed that is slower than the upper speed limit and/or faster than the lower speed limit, the processor 116 optionally can direct the display device 118 to present a visual movement indicator 604 on the display device 118, as shown in FIGS. 6 through 11. This indicator 604 is shown as an elongated, horizontal bar having a color that can change based on the probe speed. Optionally can be shown in another way, such as a circle, square, or other shape, that changes color based on probe speed, text that changes based on probe speed, or the like. The indicator 604 can increase in length as more image data is acquired. For example, the indicator 604 may only extend below the portions 600, 700, 800, 900, 1000, 1100 of the image data as these portions 600, 700, 800, 900, 1000, 1100 are acquired. The indicator 604 may only extend below the portion 600 of the image data as the portion 600 is acquired and displayed. Then, the indicator 604 can increase in length to below both the portion 600 and the portion 700 of the image data as the portion 700 is acquired and displayed, and so on.

At 410, a warning of the probe speed is presented to the operator of the imaging probe. The processor 116 can direct the display device 118 to visually present the warning, such as by displaying one or more graphical icons and/or text, activating a light, or the like. In one embodiment, the processor 116 can direct the display device 118 to change a color or other characteristic (e.g., brightness, shape, size, etc.) of the indicator 604 responsive to the probe speed being too fast or too slow. For example, the indicator 604 may be shown in a green color while the probe 106 is moving at a speed between the lower and upper speed limits. Responsive to the processor 116 determining that the probe 106 is moving too fast or too slow (relative to the limits), the processor 116 can direct the display device 118 to change a characteristic of the indicator 604, such as by changing the color of a portion of the indicator 604.

For example, a first portion 606 of the indicator 604 can be shown below or otherwise near the portion 600 of the image data as the portion 600 of the image data is shown on the display device 118, as shown in FIG. 6. This first portion 606 may be shown in a first color (e.g., green) because the probe 106 was moved at an acceptable speed while the first portion 600 of the image data was acquired by the probe 106 (e.g., faster than the lower speed limit but slower than the upper speed limit). The indicator 604 may be elongated to include a second portion 706 (shown in FIG. 7) as the second portion 700 of the image data is shown on the display device 118. This second portion 706 may be shown in the same first color (e.g., green) because the probe 106 was moved at an acceptable speed while the second portion 700 of the image data was acquired by the probe 106.

But, the probe 106 may be moved too rapidly or too slowly during at least part of the time that the third portion 800 (shown in FIG. 8) of the image data is obtained. To provide the warning to the operator, the processor 116 can direct the display device 118 to change the color or other characteristic of the indicator 604 responsive to determining that the probe 106 is moving too quickly or too slowly. For example, the processor 116 can direct the display device 118 to display a third portion 806 of the indicator 604 in a different color (e.g., yellow). This change in color can inform the operator of the imaging system 100 that a segment 802 of the portion 800 of the image data was acquired while the probe 106 was moved too quickly or too slowly over a corresponding area over the person 204. The operator can then move the probe 106 back over the corresponding area of the person 204 to acquire additional image data for this area where the probe 106 previously was moved too quickly or too slowly.

In one embodiment, the color or other characteristic of the indicator 604 can change based on or responsive to a change in direction in which the probe 106 is moved relative to the person 204 being imaged. The operator may move the probe 106 in one direction along the person 204, such as toward the head of the person 204, during imaging of the lungs and ribs. But, the operator may desire to stop and move the probe 106 back away from the head of the person 204. For example, the operator may wish to obtain additional image data of one or more intercostal regions of the person 204. This may occur responsive to the indicator 604 informing the operator that the probe 106 was moved too quickly over a previously imaged intercostal region, responsive to the operator saw a potential pathological structure in a previously imaged intercostal region, or responsive to one or more other events. The operator may begin moving the probe 106 back over a previously imaged portion of the person 204, and the processor 116 can detect this reversal of movement as a change to negative speed of movement of the probe 106. The processor 116 can detect this change in movement based on the image data that is acquired (as described above), or based on sensor output (e.g., output from an accelerometer coupled with the probe 106). The processor 116 can direct the display device 118 to change the color or other characteristic of the indicator 604 responsive to detecting the change or reversal of direction of the movement of the probe 106, such as by changing the color of the indicator 604 to blue or red (or another color).

Optionally, the notification that is displayed can represent an amount of noise in the image data. The processor 116 can examine characteristics of the image data (e.g., pixel intensities, brightness, colors, etc.) to determine the amount of noise in the image data. For example, the processor 116 can calculate increased amounts of noise responsive to larger and/or more frequent changes in the pixel brightness in the image data and can calculate smaller amounts of noise responsive to smaller and/or less frequent changes in the pixel brightness in the image data. The processor 116 can compare the calculated amount of noise to one or more noise thresholds, and can direct the display device 118 to display or change a display of an indicator (e.g., the indicator 604) to indicate the noise. For example, the indicator 604 may change to the color red responsive to the amount of noise increasing above the threshold.

Optionally, the notification that is displayed can indicate whether a sweep of the ultrasound probe 106 misses a zone of interest in the person 204 and/or extends outside of a zone of interest in the person 204. A zone of interest can be one or more internal volumes of the person 204 that is sought to be imaged using the probe 106. For example, a zone of interest can include several (or all) intercostal spaces in one lung of the person 204, can include several (or all) ribs of one lung of the person 204, or the like. The processor 116 can automatically identify ribs and/or intercostal spaces in the ultrasound image data based on changes in the characteristics of the ultrasound image data, as described herein. The processor 116 can receive input (e.g., from the operator) of which intercostal spaces and/or ribs are sought to be imaged, and optionally whether the imaging will begin from a proximal or distal location along the person 204 (e.g., closer to the head or feet of the person 204). The processor 116 can then automatically identify and count the number of intercostal spaces and/or ribs to determine whether ultrasound image data of the intercostal spaces and/or ribs sought to be imaged are obtained by the imaging probe 106. For example, if the operator indicates that he or she desires to image the third intercostal space of a lung of the person 204, then the processor 116 can count the number of intercostal spaces that are imaged by the probe 106 to determine whether this third intercostal space is shown in the image data. If the desired zone of interest (e.g., the third intercostal space) is not imaged, then the processor 116 can change the indicator 604 (or present other information) on the display device 118 to inform the operator that the zone of interest was not imaged.

The processor 116 can determine if the probe 106 is extending outside of a location where the zone of interest is being imaged and provide a notification to the operator. For example, during imaging of a lung, the operator may sweep the probe 106 to a location that results in the ultrasound image data showing other volumes in the person 204, such as a liver, stomach, or the like. To avoid ultrasound image data of volumes other than a zone of interest being imaged and confused with the intercostal spaces or ribs of the person 204, the processor 116 can determine from where the ultrasound image data is being obtained. If the ultrasound image data is obtained from outside of a zone of interest (e.g., outside of a lung or ribs or a person 204), then the processor 116 can direct the display device 118 to change the indicator 604 (or present other information) to inform the operator. The processor 116 can determine where the image data is acquired from based on the characteristics of the image data. For example, the processor 116 can count the number of ribs and/or intercostal spaces appearing in the image data and, when all ribs or intercostal spaces are obtained and the probe 106 continues to be moved, the processor 116 can determine that the image data is acquired outside of the zone of interest.

Optionally, the notification that is displayed can indicate whether the quality of the ultrasound image data falls below one or more thresholds. For example, the processor 116 can examine characteristics of the image data to determine whether the darkness or brightness of one or more pixels representing a shadow of a rib are too bright (e.g., brighter than a threshold associated with rib shadows), whether the darkness or brightness of one or more pixels representing an intercostal space are too dark (e.g., darker than a threshold associated with intercostal spaces), or the like. As another example, the processor 116 can examine characteristics of the image data to determine whether the spacing (e.g., distance) between neighboring ribs of the person 204 is too small or too large. If the operator is moving the probe 106 too quickly or the quality of the image data is poor (e.g., the signal-to-noise ratio is too small), then the processor 116 may calculate larger or smaller distances between ribs. For example, these distances may be larger or smaller than distances associated with likely inter-rib distances associated with the person 204. The processor 116 can compare the inter-rib distances with a variable range of distances. This variable distance range can change based on the age of the person 204. If the calculated inter-rib distance is outside of the range (e.g., the calculated inter-rib distances are too long or are too short to be distances between the ribs of the person 204), then the processor 116 can change the indicator 604 (or present other information) on the display device 118 to inform the operator that the quality of the image data is poor, and optionally that the operator should control the probe 106 to acquire additional image data.

Returning to the description of the flowchart of the method 400 shown in FIG. 4, at 412, a determination is made as to whether one or more segments of interest in the combined image data are selected. The operator of the imaging system 100 can select a segment of interest 610 in one or more views of the combined image data 602 by touching a portion of the display device 118 that corresponds to a segment of interest 610 or by using another input device to select the portion of the combined image data 602 that corresponds with a segment of interest 610.

If a segment of interest 610 (or several segments of interest 610) are selected, then the processor 116 can change how the combined image data 602 is displayed. As a result, flow of the method 400 can proceed toward 414. But, if no segment of interest 610 is selected, then the processor 116 may not change how the combined image data 602 is displayed. As a result, the method 400 can terminate or return toward one or more other operations of the method 400.

At 414, one or more segments of interest in the combined image data are dynamically displayed, and one or more other segments of interest in the combined image data are statically displayed. For example, the processor 116 can direct the display device 118 to display a video of the image data corresponding with the segment of interest 610 that was selected at 412. The processor 116 also can direct the display device 118 to display still images of the image data corresponding with segments of interest 610 (e.g., all the remaining, non-selected segments of interest 610) responsive to the selection of a segment of interest at 412.

The operator can view the video of the selected segment of interest 610 and the stationary images of the other segments of interest 610 and subsequently select another segment of interest 610. Responsive to selecting another segment of interest 610, the processor 116 can direct the display device 118 to present a video of the other selected segment of interest 610 and present still images of the other segments of interest 610. This can allow for the operator to change which segments of interest 610 are shown as moving videos and which segments of interest 610 are shown as still images at different times.

Alternatively, the processor 116 can direct the display device 118 to present the selected segment of interest 610 as a still image and the other segments of interest 610 (that were not selected) as moving videos. Flow of the method 400 can then terminate or can return toward one or more other operations of the method 400, such as 412.

Alternatively, the processor 116 can direct the display device 118 to present multiple segments of interest 610, or all segments of interest 610, as moving videos. For example, the processor 116 can dynamically display all segments of interest responsive to receive of user input (e.g., at 412). Optionally, the method 400 can automatically present all or multiple segments of interest 610 as moving videos without or regardless of user input that is received.

In one embodiment, a method includes acquiring ultrasound image data from moving an ultrasound probe over a body of a person, automatically dividing the ultrasound image data into segments of interest based on where the ultrasound image data was acquired, and displaying a panoramic view of the ultrasound image data that includes two or more of the segments of interest with at least one of the segments of interest displayed as a video.

Optionally, displaying the panoramic view of the ultrasound image data includes displaying the at least one of the segments of interest as the video and at least one other segment of the segments of interest statically displayed as a frame of the ultrasound image data.

Optionally, displaying the panoramic view of the ultrasound image data includes displaying two or more of the segments of interest as videos.

Optionally, the method also includes temporally synchronizing the ultrasound image data of the two or more segments of interest that are displayed as the videos in the panoramic view.

Optionally, the ultrasound image data of the two or more segments of interest are temporally synchronized with a respiratory cycle of the person.

Optionally, temporally synchronizing the ultrasound image data for the two or more segments of interest includes temporally scaling the ultrasound image data for at least one of the segments of interest due to a change in the respiratory cycle of the patient.

Optionally, the ultrasound image data is acquired while moving the ultrasound probe in a first direction and then in a different, second direction.

Optionally, the ultrasound image data represents a lung and ribs of the person, and the segments of interest are inter-rib segments of interest located between the ribs of the person.

Optionally, the method also includes measuring movement of pleura in the ultrasound image data, and calculating a respiratory cycle timing of the patient based on the movement of the pleura that is measured in the ultrasound image data.

Optionally, the method also includes receiving a movement indication that changes a graphical location of the ultrasound image data associated with one or more of the segments of interest, and re-arranging locations of the one or more segments of interest associated with the graphical location that is changed in the panoramic view responsive to and based on receiving the movement indication.

Optionally, the method also includes automatically examining frames of the ultrasound image data for at least one of the segments of interest to identify one or more regions of interest, and automatically displaying the frames of the ultrasound image data having the one or more regions of interest that are identified in the panoramic view.

Optionally, the method also includes determining one or more of a speed or a direction at which the ultrasound probe is moved over the person based on the ultrasound image data.

Optionally, the method also includes displaying a notification to an operator of the ultrasound probe of one or more of the speed at which the ultrasound probe is moved being faster than an upper designated speed limit, the speed at which the ultrasound probe is moved being slower than a lower designated speed limit, or a change in the direction in which the ultrasound probe is moved over the person.

Optionally, the method also includes displaying one or more graphical anatomical features with the video of the ultrasound image data in the panoramic view. The one or more graphical anatomical features can represent locations of one or more anatomical bodies of the person of which the image data is acquired.

Optionally, the method also includes determining that the ultrasound probe has been moved and is no longer acquiring the ultrasound image data of a zone of interest within the body of the person, and displaying an indicator that notifies an operator of the ultrasound probe that the ultrasound probe is no longer acquiring the ultrasound image data of the zone of interest.

Optionally, the ultrasound image data shows ribs of the person, and the method also can include determining a quality of detection of one or more of the ribs based on the ultrasound image data, and displaying an indicator that notifies an operator of the ultrasound probe that the quality of detection is below a threshold based on a characteristic of the ultrasound image data showing a shadow of the one or more ribs or a spacing between two or more of the ribs being outside of a designated range.

In one embodiment, a system includes an ultrasound probe configured to acquire ultrasound image data while moving over a body of a person, and one or more processors configured to automatically divide the ultrasound image data into segments of interest based on where the ultrasound image data was acquired. The one or more processors also are configured to direct a display device to display a panoramic view of the ultrasound image data that includes two or more of the segments of interest with at least one of the segments of interest displayed as a video.

Optionally, the one or more processors are configured to direct the display device to display the panoramic view of the ultrasound image data by displaying the at least one of the segments of interest as the video and at least one other segment of the segments of interest statically displayed as a frame of the ultrasound image data.

Optionally, the one or more processors are configured to direct the display device to display the panoramic view with two or more of the segments of interest as videos.

In one embodiment, a method includes acquiring ultrasound image data from longitudinally moving an ultrasound probe over a person, automatically dividing the ultrasound image data into segments based on where the ultrasound image data is acquired in the person, and displaying a panoramic view of the segments of the ultrasound image data. The panoramic view includes at least one of the segments of the ultrasound image data displayed as a video.

Optionally, displaying the panoramic view includes displaying at least two of the segments of the ultrasound image data as videos.

Optionally, displaying the panoramic view includes also displaying at least one of the segments of the ultrasound image data as a static frame concurrent with displaying the at least one of the segments of the ultrasound image data as a video.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements that do not have that property.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method comprising:
acquiring ultrasound image data from moving an ultrasound probe over a body of a person;
automatically dividing the ultrasound image data into segments of interest based on where the ultrasound image data was acquired; and
displaying a panoramic view of the ultrasound image data that includes two or more of the segments of interest with at least one of the segments of interest displayed as a video.

2. The method of claim 1, wherein displaying the panoramic view of the ultrasound image data includes displaying the at least one of the segments of interest as the video and at least one other segment of the segments of interest statically displayed as a frame of the ultrasound image data.

3. The method of claim 1, wherein displaying the panoramic view of the ultrasound image data includes displaying two or more of the segments of interest as videos.

4. The method of claim 3, further comprising:
temporally synchronizing the ultrasound image data of the two or more segments of interest that are displayed as the videos in the panoramic view.

5. The method of claim 4, wherein the ultrasound image data of the two or more segments of interest are temporally synchronized with a respiratory cycle of the person.

6. The method of claim 4, wherein temporally synchronizing the ultrasound image data for the two or more segments of interest includes temporally scaling the ultrasound image data for at least one of the segments of interest due to a change in the respiratory cycle of the patient.

7. The method of claim 1, wherein the ultrasound image data is acquired while moving the ultrasound probe in a first direction and then in a different, second direction.

8. The method of claim 1, wherein the ultrasound image data represents a lung and ribs of the person, and the segments of interest are inter-rib segments of interest located between the ribs of the person.

9. The method of claim 1, further comprising:
measuring movement of pleura in the ultrasound image data; and
calculating a respiratory cycle timing of the patient based on the movement of the pleura that is measured in the ultrasound image data.

10. The method of claim 1, further comprising:
receiving a movement indication that changes a graphical location of the ultrasound image data associated with one or more of the segments of interest; and
re-arranging locations of the one or more segments of interest associated with the graphical location that is changed in the panoramic view responsive to and based on receiving the movement indication.

11. The method of claim 1, further comprising:
automatically examining frames of the ultrasound image data for at least one of the segments of interest to identify one or more regions of interest; and
automatically displaying the frames of the ultrasound image data having the one or more regions of interest that are identified in the panoramic view.

12. The method of claim 1, further comprising:
determining one or more of a speed or a direction at which the ultrasound probe is moved over the person based on the ultrasound image data.

13. The method of claim 12, further comprising:
displaying a notification to an operator of the ultrasound probe of one or more of the speed at which the ultrasound probe is moved being faster than an upper designated speed limit, the speed at which the ultrasound probe is moved being slower than a lower designated speed limit, or a change in the direction in which the ultrasound probe is moved over the person.

14. The method of claim 1, further comprising:
displaying one or more graphical anatomical features with the video of the ultrasound image data in the panoramic view, the one or more graphical anatomical features representing locations of one or more anatomical bodies of the person of which the image data is acquired.

15. The method of claim 1, further comprising:
determining that the ultrasound probe has been moved and is no longer acquiring the ultrasound image data of a zone of interest within the body of the person; and
displaying an indicator that notifies an operator of the ultrasound probe that the ultrasound probe is no longer acquiring the ultrasound image data of the zone of interest.

16. The method of claim 1, wherein the ultrasound image data shows ribs of the person, and further comprising:
determining a quality of detection of one or more of the ribs based on the ultrasound image data; and
displaying an indicator that notifies an operator of the ultrasound probe that the quality of detection is below a threshold based on a characteristic of the ultrasound image data showing a shadow of the one or more ribs or a spacing between two or more of the ribs being outside of a designated range.

17. A system comprising:
an ultrasound probe configured to acquire ultrasound image data while moving over a body of a person; and
one or more processors configured to automatically divide the ultrasound image data into segments of interest based on where the ultrasound image data was acquired, the one or more processors also configured to direct a display device to display a panoramic view of the ultrasound image data that includes two or more of the segments of interest with at least one of the segments of interest displayed as a video.

18. The system of claim 17, wherein the one or more processors are configured to direct the display device to display the panoramic view of the ultrasound image data by displaying the at least one of the segments of interest as the video and at least one other segment of the segments of interest statically displayed as a frame of the ultrasound image data.

19. The system of claim 17, wherein the one or more processors are configured to direct the display device to display the panoramic view with two or more of the segments of interest as videos.

20. A method comprising:
acquiring ultrasound image data from longitudinally moving an ultrasound probe over a person;
automatically dividing the ultrasound image data into segments based on where the ultrasound image data is acquired in the person; and
displaying a panoramic view of the segments of the ultrasound image data, the panoramic view including at least one of the segments of the ultrasound image data displayed as a video.

21. The method of claim 20, wherein displaying the panoramic view includes displaying at least two of the segments of the ultrasound image data as videos.

22. The method of claim 20, wherein displaying the panoramic view includes also displaying at least one of the segments of the ultrasound image data as a static frame concurrent with displaying the at least one of the segments of the ultrasound image data as a video.
